(19) United States
(12) Patent Application Publication
(10) Pub. No.: US 2024/0173289 A1
(43) Pub. Date: May 30, 2024

(54) USE OF OXYMETHYL-MODIFIED DERIVATIVE OF QUERCETIN IN PREPARATION OF DRUG FOR INHIBITING TUMOR CELL PROLIFERATION

(71) Applicant: ZHEJIANG UNIVERSITY, Hangzhou, Zhejiang (CN)

(72) Inventors: Chongde SUN, Hangzhou, Zhejiang (CN); Yue WANG, Hangzhou, Zhejiang (CN); Yunyi CHEN, Hangzhou, Zhejiang (CN); Jinping CAO, Hangzhou, Zhejiang (CN); Xian LI, Hangzhou, Zhejiang (CN)

(73) Assignee: ZHEJIANG UNIVERSITY, Hangzhou, Zhejiang (CN)

(21) Appl. No.: 18/551,314

(22) PCT Filed: Apr. 20, 2022
(86) PCT No.: PCT/CN2022/087786

§ 371 Date: Sep. 19, 2023

(30) Foreign Application Priority Data
Dec. 22, 2021 (CN) 202111578422.3

(51) Int. Cl. A61K 31/352; A61P 35/00

(52) U.S. Cl. CPC A61K 31/352; A61P 35/00

(57) ABSTRACT

The present disclosure relates to use of an oxymethyl-modified derivative of quercetin in preparation of a drug for inhibiting tumor cell proliferation, and belongs to the technical field of drug preparation. The present disclosure provides use of an oxymethyl-modified derivative of quercetin in preparation of a drug for inhibiting tumor cell proliferation. The oxymethyl-modified derivative of quercetin may inhibit tumor cell proliferation, inhibit proliferation of a transplanted tumor in vivo, inhibit tumor cell metastasis, and induce tumor cell apoptosis.

### CROSS REFERENCE TO RELATED APPLICATION

The present application claims priority to the Chinese Patent Application No. 202111578422.3, filed with the China National Intellectual Property Administration (CNIPA) on Dec. 22, 2021, and entitled “USE OF OXYMETHYL-MODIFIED DERIVATIVE OF QUERCETIN IN PREPARATION OF DRUG FOR INHIBITING TUMOR CELL PROLIFERATION”, which is incorporated herein by reference in its entirety.

### TECHNICAL FIELD

The present disclosure relates to the technical field of drug preparation, in particular to use of an oxymethyl-modified derivative of quercetin in preparation of a drug for inhibiting tumor cell proliferation.

### BACKGROUND

Cancer is one of the diseases with the highest death rate. Due to changes in living habits and increased exposure to carcinogens, the occurrence of cancer has gradually become younger and more common. Risk factors associated with cancer include unhealthy diet, exposure to pollutants, stress, and inflammation. Traditional radiotherapy and chemotherapy can seriously reduce the life quality of patients, while the new targeted therapy is extremely expensive and currently difficult to promote on a large scale.

Epidemiological studies have shown that the intake of fruits and vegetables rich in natural products has a significant effect on inhibiting the development of gastric cancer. Bioactive compounds of natural origin have better biosafety and less impact on quality of life than existing clinical cancer drugs. Therefore, it is highly necessary to screen natural products with anticancer or adjuvant therapeutic effects.

### SUMMARY

An object of the present disclosure is to provide use of an oxymethyl-modified derivative of quercetin in preparation of a drug for inhibiting tumor cell proliferation. The oxymethyl-modified derivative of quercetin may inhibit tumor cell proliferation, inhibit proliferation of a transplanted tumor in vivo, inhibit tumor cell metastasis, and induce tumor cell apoptosis.

The present disclosure provides use of an oxymethyl-modified derivative of quercetin in preparation of a drug for inhibiting tumor cell proliferation.

The present disclosure further provides use of an oxymethyl-modified derivative of quercetin in preparation of a drug for inhibiting proliferation of a transplanted tumor in vivo.

The present disclosure further provides use of an oxymethyl-modified derivative of quercetin in preparation of a drug for inhibiting tumor cell metastasis.

The present disclosure further provides use of an oxymethyl-modified derivative of quercetin in preparation of a drug for inducing tumor cell apoptosis.

In one embodiment, the oxymethyl-modified derivative of quercetin includes quercetin 3-methyl ether and/or quercetin 3,3′-dimethyl ether.

In one embodiment, the tumor cell is a cell selected from the group consisting of osteosarcoma, liver cancer, breast cancer, gastric cancer, cervical cancer, lung cancer, intestinal cancer, glioma, prostate cancer, and thyroid cancer.

The present disclosure provides use of an oxymethyl-modified derivative of quercetin in preparation of a drug for inhibiting tumor cell proliferation. In the present disclosure, it is found that modifying quercetin at a specific site can significantly improve the ability of this substance to inhibit the proliferation of tumor cells. For example, after methylation at the 3-position and 3,3′-position, the half-inhibitory concentration of resulting derivatives on the tumor cell proliferation is significantly reduced. The oxymethyl-modified derivative of quercetin may inhibit tumor cell proliferation, inhibit proliferation of a transplanted tumor in vivo, inhibit tumor cell metastasis, and induce tumor cell apoptosis. The test results show that quercetin 3-methyl ether has an inhibitory effect on all tested tumor cell lines, which is 2.4 to 3.6 times higher than that of quercetin. Compared with the quercetin 3-methyl ether, the inhibitory effect of quercetin 3,3′-dimethyl ether is further improved by 4.3 to 14.5 times. There is the strongest inhibitory effect on HepG2, with a half-inhibitory concentration of only 5.85 mg/L. The quercetin 3,3′-dimethyl ether has an in vitro proliferation inhibitory effect on 15 kinds of cancer cells, and shows biological safety on 4 kinds of normal cells; this derivative has an effect of inhibiting the proliferation of HepG2 mouse transplanted tumor in vivo, and an effect of inhibiting the metastasis capacity of HepG2 cells. This derivative shows a significant apoptosis-inducing effect on all 14 kinds of cells.

### BRIEF DESCRIPTION OF THE DRAWINGS

FIG. 1 shows a liquid phase diagram of the quercetin 3,3′-dimethyl ether provided by the present disclosure;

FIG. 2 shows a mass spectrogram of the quercetin 3,3′-dimethyl ether provided by the present disclosure;

FIG. 3 shows a comparison result of an in vitro proliferation inhibitory effect of the quercetin 3,3′-dimethyl ether provided by the present disclosure on 15 kinds of cancer cells and 4 kinds of normal cells;

FIG. 4 shows an inhibitory effect of the quercetin 3,3′-dimethyl ether provided by the present disclosure on proliferation of transplanted tumors in a HepG2 mouse in vivo, where A is a tumor volume, B is a tumor weight, C is a tumor photo, D is a body weight of the mouse, E is a liver index, F is a spleen index, and G is a renal index;

FIG. 5 shows an inhibition result of the quercetin 3,3′-dimethyl ether provided by the present disclosure on the metastasis capacity of HepG2 cells.

### DETAILED DESCRIPTION OF THE EMBODIMENTS

The present disclosure provides use of an oxymethyl-modified derivative of quercetin in preparation of a drug for inhibiting tumor cell proliferation. In the present disclosure, the oxymethyl-modified derivative of quercetin includes quercetin 3-methyl ether and/or quercetin 3,3′-dimethyl ether. The tumor cell is a cell selected from the group consisting of osteosarcoma, liver cancer, breast cancer, gastric cancer, cervical cancer, lung cancer, intestinal cancer, glioma, prostate cancer, and thyroid cancer. In the present disclosure, it is found that modifying quercetin at a specific site may significantly improve the ability of this substance to inhibit the proliferation of tumor cells. For example, after methylation at the 3-position and 3,3′-position, the half-inhibitory concentration of resulting derivatives on the tumor cell proliferation is significantly reduced. There is no special limitation on sources of the quercetin, quercetin 3-methyl ether, and quercetin 3,3′-dimethyl ether, and conventional commercially available products well known to those skilled in the art may be used.

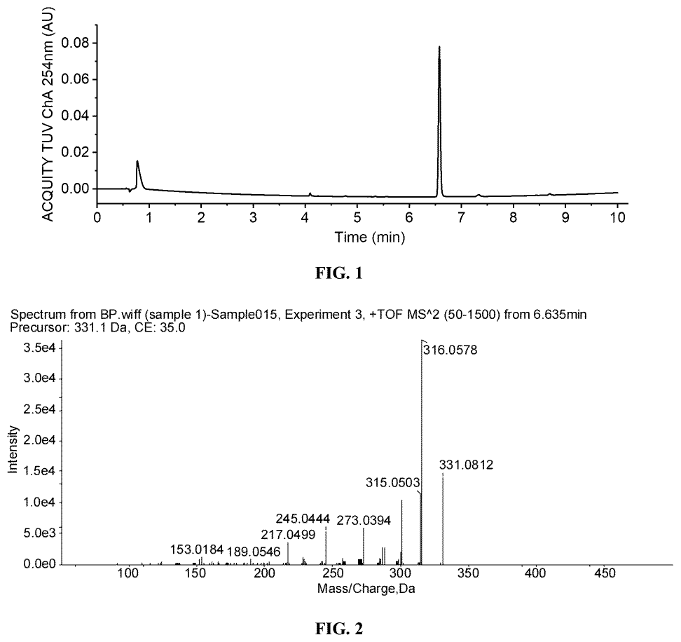

The quercetin 3,3′-dimethyl ether is a derivative of common flavonol quercetin. In the present disclosure, it is found that the quercetin 3,3′-dimethyl ether has an extensive tumor growth inhibitory ability. The quercetin 3,3′-dimethyl ether has a CAS number of 4382-17-6, a structure shown in formula I, a liquid phase diagram shown in FIG. 1, and a mass spectrum shown in FIG. 2. The quercetin 3,3′-dimethyl ether not only has the ability to significantly inhibit tumor proliferation, but also has weak toxicity to normal cells, and is safe when injected into the intraperitoneal cavity.

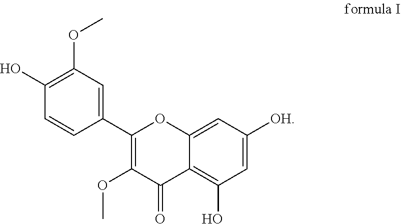 [SMILES: COc1cc(-c2oc3cc(O)cc(O)c3c(=O)c2OC)ccc1O]

The present disclosure further provides use of an oxymethyl-modified derivative of quercetin in preparation of a drug for inhibiting proliferation of a transplanted tumor in vivo. In the present disclosure, the oxymethyl-modified derivative of quercetin includes quercetin 3-methyl ether and/or quercetin 3,3′-dimethyl ether. The tumor cell is a cell selected from the group consisting of osteosarcoma, liver cancer, breast cancer, gastric cancer, cervical cancer, lung cancer, intestinal cancer, glioma, prostate cancer, and thyroid cancer.

The present disclosure further provides use of an oxymethyl-modified derivative of quercetin in preparation of a drug for inhibiting tumor cell metastasis. In the present disclosure, the oxymethyl-modified derivative of quercetin includes quercetin 3-methyl ether and/or quercetin 3,3′-dimethyl ether. The tumor cell is a cell selected from the group consisting of osteosarcoma, liver cancer, breast cancer, gastric cancer, cervical cancer, lung cancer, intestinal cancer, glioma, prostate cancer, and thyroid cancer.

The present disclosure further provides use of an oxymethyl-modified derivative of quercetin in preparation of a drug for inducing tumor cell apoptosis. In the present disclosure, the oxymethyl-modified derivative of quercetin includes quercetin 3-methyl ether and/or quercetin 3,3′-dimethyl ether. The tumor cell is a cell selected from the group consisting of osteosarcoma, liver cancer, breast cancer, gastric cancer, cervical cancer, lung cancer, intestinal cancer, glioma, prostate cancer, and thyroid cancer.

The use of an oxymethyl-modified derivative of quercetin in preparation of a drug for inhibiting tumor cell proliferation provided by the present disclosure will be further described in detail below in conjunction with examples, and the technical solutions of the present disclosure include but are not limited to the following examples.

### Example 1

Comparison of In Vitro Cell Proliferation Inhibition of Quercetin, Quercetin 3-Methyl Ether, and Quercetin 3,3′-Dimethyl Ether

Method:

Osteosarcoma cell line 143B, HOS, and U2, liver cancer cell line HepG2, breast cancer cell line MDA-MB-231 and T47D, gastric cancer cell line AGS, BGC-823, and SGC-7901, cervical cancer cells Hela, lung cancer cells A549, intestinal cancer cells Caco2, mouse glioma cells BV-2, human prostate cancer cells PC3, human thyroid cancer cell line BCPAP, human normal liver cell line L02, human normal gastric cell line GES, human umbilical vein epithelial cells HUVEC, and human embryonic lung fibroblasts WI-38 were cultured in a DMEM medium containing 10% fetal bovine serum and 1×HEPES in a humid incubator (37° C., 5% CO₂). The cells were passaged with trypsin-EDTA in logarithmic phase. Certain density of cells (AGS, T47D, Caco2, BCPAP, GES, HUVEC 1×10⁵ cells per well; HepG2, MDA-MB-231, BGC-823, A549, PC3, L02 8×10⁴ cells per well; 143B, HOS, U2, SGC-7901, Hela, BV-2, 5×10⁴ cells per well; WI-38, 3×10⁵ cells per well) were inoculated in a 96-well plate, and cultured for 24 h before treatment. After replacing a fresh medium, 12.5 mg/L to 400 mg/L of quercetin, quercetin 3-methyl ether, and quercetin 3,3′-dimethyl ether dissolved with DMSO were added into the medium (a final volume ratio was 0.1%, and the DMSO was used as a positive control). After incubation for 48 h, a complete medium was replaced with a serum-free medium containing 10% CCK-8 reagent. The absorbance at 450 nm and 620 nm was measured separately after the cells were incubated for 1 h. Each experiment was repeated three times independently.

Results were shown in Table 1.

TABLE 1
Comparative results of in vitro cell proliferation inhibition of quercetin,
quercetin 3-methyl ether, and quercetin 3,3′-dimethyl ether

| | | IC₅₀ (mg/L) | |
|---|---|---|---|
| | | Quercetin 3- | Quercetin 3,3′- |
| Cell line | Quercetin | methyl ether | dimethyl ether |
| 143B | / | 210.34 | 21.62 |
| HOS | / | 130.84 | 18.60 |
| U2 | / | 377.08 | 49.47 |
| HepG2 | 155.01 | 58.45 | 5.85 |
| MDA-MB-231 | 330.65 | 92.14 | 11.39 |
| T47D | / | 154.72 | 13.33 |
| SGC7901 | / | 123.94 | 28.38 |
| BGC823 | / | 192.55 | 26.07 |
| AGS | 329.40 | 117.04 | 15.83 |
| Hela | 195.91 | 82.34 | 11.13 |
| A549 | / | 252.27 | 17.38 |
| Caco2 | / | 215.61 | 21.39 |
| BV2 | / | 288.00 | 21.12 |
| PC3 | / | 368.40 | 31.29 |
| BCPAP | 391.23 | 157.67 | 12.30 |

Note:
“/” meant that the IC₅₀ value could not be calculated.

As shown in Table 1, after modifying quercetin with 3- and 3,3′-oxymethyl groups, the ability to inhibit the proliferation of various tumor cells is greatly improved, and the half-inhibitory concentration is significantly reduced. The quercetin only has inhibitory effects on HepG2, MDA-MB-231, AGS, Hela, and BCPAP. The quercetin 3-methyl ether has an inhibitory effect on all detected tumor cell lines, which is 2.4 to 3.6 times higher than that of the quercetin. Compared with the quercetin 3-methyl ether, the quercetin 3,3′-dimethyl ether has an inhibitory effect further increases by 4.3 to 14.5 times, and shows the strongest inhibitory effect on HepG2, with a half-inhibitory concentration of only 5.85 mg/L.

### Example 2

Comparison of In Vitro Proliferation Inhibitory Effect of the Quercetin 3,3′-Dimethyl Ether on 15 Kinds of Cancer Cells and 4 Kinds of Normal Cells

Method:

Osteosarcoma cell line 143B, HOS, and U2, liver cancer cell line HepG2, breast cancer cell line MDA-MB-231 and T47D, gastric cancer cell line AGS, BGC-823, and SGC-7901, cervical cancer cells Hela, lung cancer cells A549, intestinal cancer cells Caco2, mouse glioma cells BV-2, human prostate cancer cells PC3, human thyroid cancer cell line BCPAP, human normal liver cell line L02, human normal gastric cell line GES, human umbilical vein epithelial cells HUVEC, and human embryonic lung fibroblasts WI-38 were cultured in a DMEM medium containing 10% fetal bovine serum and 1×HEPES in a humid incubator (37° C., 5% CO₂). The cells were passaged with trypsin-EDTA in logarithmic phase. Certain density of cells (AGS, T47D, Caco2, BCPAP, GES, HUVEC 1×10⁵ cells per well; HepG2, MDA-MB-231, BGC-823, A549, PC3, L02 8×10⁴ cells per well; 143B, HOS, U2, SGC-7901, Hela, BV-2, 5×10⁴ cells per well; WI-38, 3×10⁵ cells per well) were inoculated in a 96-well plate, and cultured for 24 h before treatment. After replacing a fresh medium, 12.5 mg/L to 400 mg/L, of quercetin 3,3′-dimethyl ether dissolved with DMSO was added into a medium (a final volume ratio was 0.1%, and the DMSO was used as a positive control). After incubation for 48 h, a complete medium was replaced with a serum-free medium containing 10% CCK-8 reagent. The absorbance at 450 nm and 620 nm was measured separately after the cells were incubated for 1 h. Each experiment was repeated three times independently.

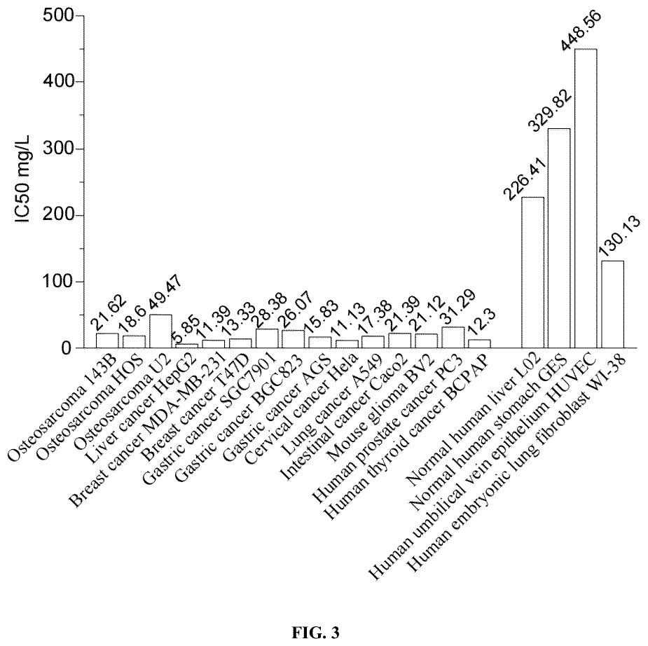

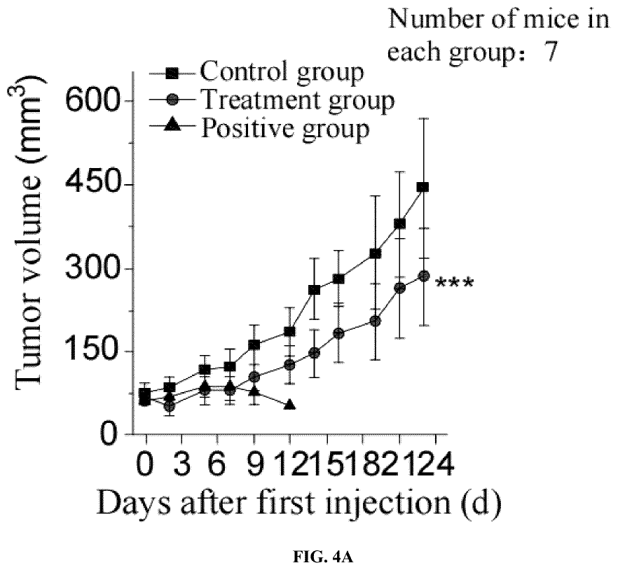

The results are shown in FIG. 3, the quercetin 3,3′-dimethyl ether exhibits a broad-spectrum inhibitory ability to various cancer cells. The half-inhibitory concentration for osteosarcoma cells is 143B: 21.62 mg/L, HOS: 18.60 mg/L, U2: 49.47 mg/L; the half-inhibitory concentration for liver cancer cells HepG2 cells is 5.85 mg/L; the half-inhibitory concentration for breast cancer cells is MDA-MB-231: 11.39 mg/L, T47D: 13.33 mg/L; the half-inhibitory concentration for gastric cancer cells is SGC7901: 28.38 mg/L, BGC823: 26.07 mg/L, AGS: 15.83 mg/L; the half-inhibitory concentration for cervical cancer Hela cells is 11.13 mg/L; the half-inhibitory concentration for lung cancer cell A549 is 17.38 mg/L; the half-inhibitory concentration for intestinal cancer cells Caco2 is 21.39 mg/L; the half-inhibitory concentration for mouse glioma BV2 is 21.12 mg/L; the half-inhibitory concentration for human prostate cancer cells PC3 is 31.29 mg/L; and the half-inhibitory concentration for human thyroid cancer cells BCPAP is 12.30 mg/L.

At the same time, in the present disclosure, it is found that the half-inhibitory concentration of quercetin 3,3′-dimethyl ether to the human normal liver cells L02, human normal gastric cells GES, human umbilical vein epithelial cells HUVEC, and human embryonic lung fibroblasts WI-38 is above 100 mg/L. This indicates that the quercetin 3,3′-dimethyl ether has a selective proliferation inhibitory effect on cancer cells, while exhibiting low toxicity to normal cells.

### Example 3

Inhibitory Effect of Quercetin 3,3′-Dimethyl Ether on Proliferation of Transplanted Tumors in HepG2 Mice In Vivo

Method:

BALB/c nude mice (5 to 6 weeks old), weighing 19 g to 22 g, were purchased from Shanghai Experimental Animal Center, Chinese Academy of Sciences. HepG2 cells were collected in a serum-free DMEM medium to make a cell suspension, and then the cell suspension was injected into an underarm area of each mouse, approximately 2.0×10⁶ cells, one site per mouse. All mice were bred in the Experimental Animal Center of Zhejiang University, under the environment of 23° C. to 25° C., humidity 50% to 60%, and cycle of 12 h light illumination/dark 12 h. After tumor formation, the mice were randomly divided into 3 groups with 7 mice in each group. The model group was given saline by intraperitoneal injection; the positive drug group was given tegafur at 10 mg/kg bw·d by intraperitoneal injection; the treatment group was intraperitoneally injected with the quercetin 3,3′-dimethyl ether at 50 mg/kg bw·d. According to the change of tumor volume, after the tumor formed, intraperitoneal injection was conducted every 1 d to 2 d, the tumor volume was measured, and the body weight of the mice was weighed. When there was a significant difference in tumor volume between different treatment groups, the mice were sacrificed by cervical dislocation, and the tumor tissue was carefully peeled off, weighed, and calculated. The formulas were: tumor formation rate (%)=(number of tumor-forming mice in treatment group/number of tumor-forming mice in inoculation group)×100; tumor inhibition rate (%)=(average tumor weight in control group (g)−average tumor weight in administration group (g))/average tumor weight in control group (g)×100. Serum, liver, spleen, and kidney were collected and weighed.

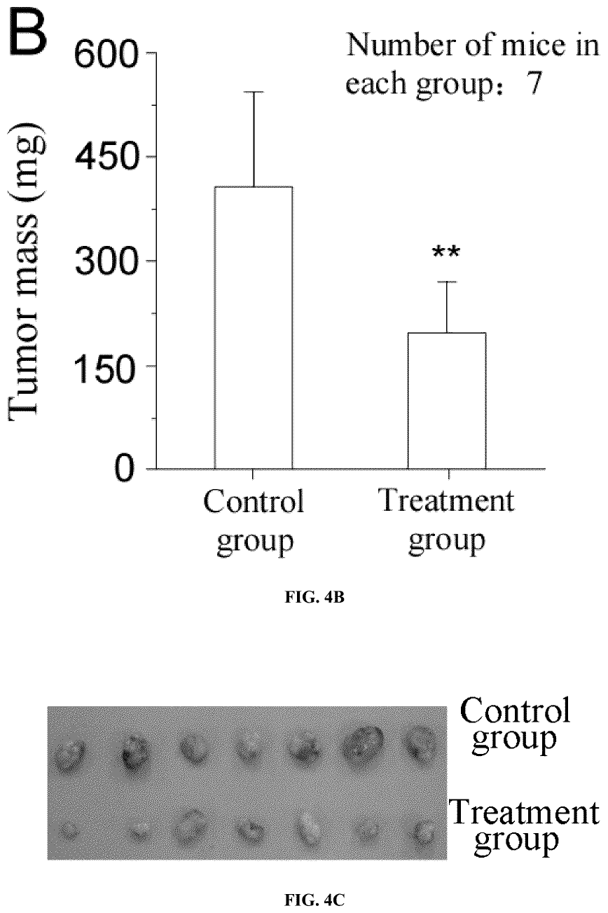

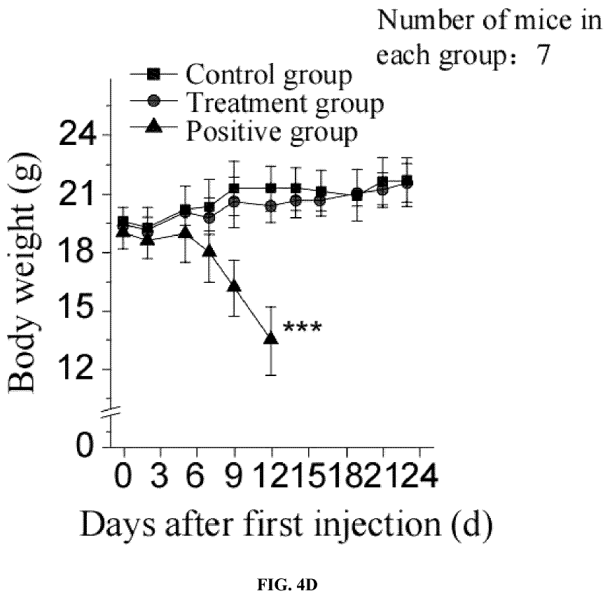

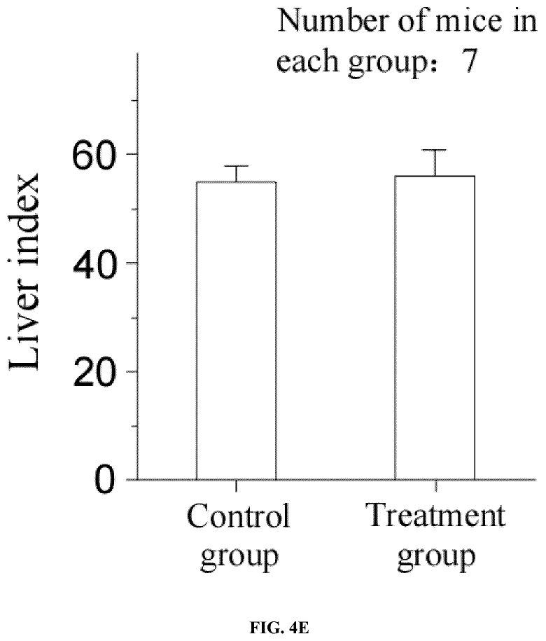

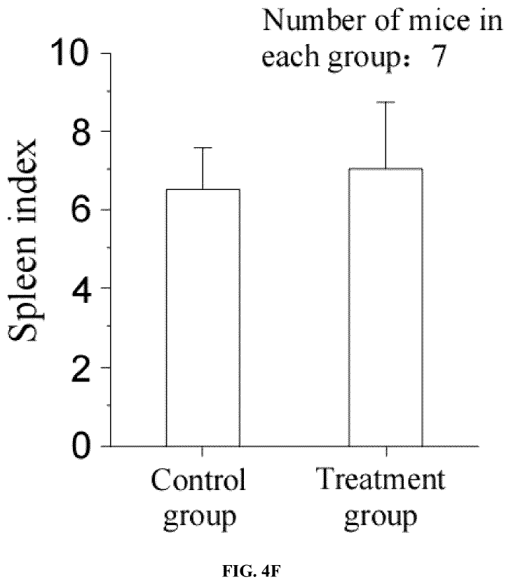

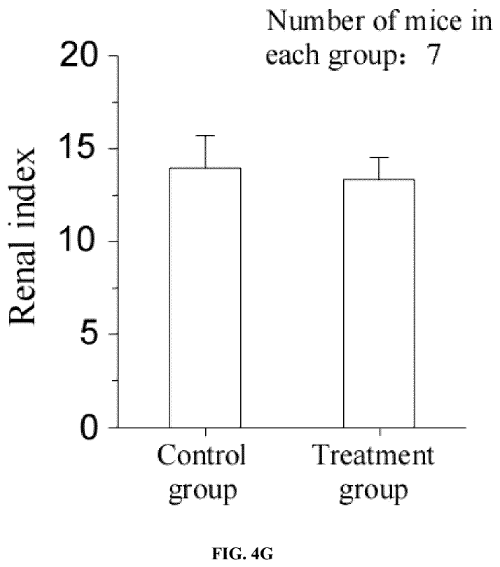

The results are shown in FIG. 4. According to FIG. 4A, from the first administration to 24 d, a total of 10 injections are conducted. At day 24, tumor volume is significantly reduced compared to the control group. The tumor volume of the control group is 443.20±123.27 mm³, while that of the group injected with quercetin 3,3′-dimethyl ether is 284.19±85.47 mm³. As shown in FIG. 4B, after the tumor is peeled off and weighed, it is found that the weight of the tumor in the control group is 407.00±134.91 mg; while the tumor weight of the group injected with quercetin 3,3′-dimethyl ether is 194.57±75.45 mg, which is 52.19% lower than that of the control group, and showed significance. FIG. 4C shows a photograph of tumors in the control group and the treatment group. The above results indicate that the quercetin 3,3′-dimethyl ether has an effect of inhibiting the proliferation of HepG2 transplanted tumors in vivo.

Meanwhile, the quercetin 3,3′-dimethyl ether exhibited biological safety. As shown in FIG. 4D, there is no significant difference in body weight between the control group and the treatment group, while the body weight of the mice in the positive drug group decreases significantly on day 12, and the mice are all dead. The liver index (FIG. 4E), spleen index (FIG. 4F), and renal index (FIG. 4G) all show that there is no significant difference between the quercetin 3,3′-dimethyl ether and the control group. The above results show that the quercetin 3,3′-dimethyl ether has biological safety.

### Example 4

Inhibition Result of the Quercetin 3,3′-Dimethyl Ether on the Metastasis Capacity of HepG2 Cells

Method:

HepG2 cells with a cell density of 5×10⁵/mL were plated on a 6-well plate (1 mL per well), added to a DMEM medium containing 10% fetal calf serum, and cultured overnight to form a monolayer of cells. A horizontal scratch was scratched on the monolayer of cells with a pipette tip of 200 μL, washed with PBS 3 times, added into a DMEM medium containing 10% fetal bovine serum, and then added with 1 mg/L of a quercetin 3,3′-dimethyl ether solution, while DMSO was used as a solvent control, and then incubated for 24 h.

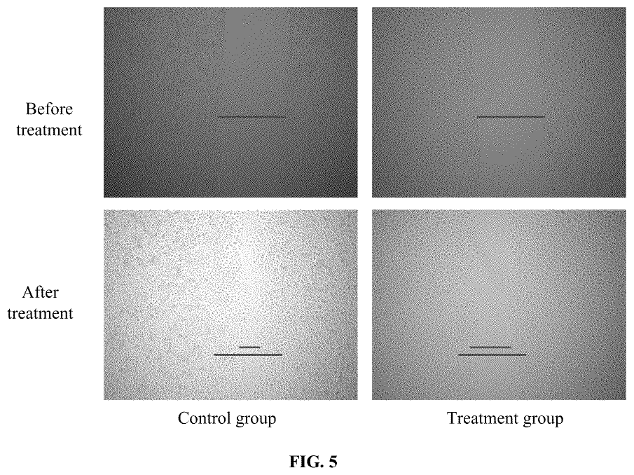

Results are shown in FIG. 5. As shown in FIG. 5, the control group and the treatment group have the same distance between cells when scratched; after 1 d of incubation, the intercellular space of the treatment group is significantly higher than that of the control group. This shows that the quercetin 3,3′-dimethyl ether has the effect of inhibiting the metastasis capacity of HepG2 cells.

### Example 5

Inhibition Result of the Quercetin 3,3′-Dimethyl Ether on the Metastasis Capacity of Tumor Cells

Method:

Cells with a certain density (6×10⁵ cells per mL for AGS, T47D, Caco2, BCPAP, GES, HUVEC; 5×10⁵ cells per mL for HepG2, MDA-MB-231, BGC-823, A549, PC3, L02; 3×10⁵ cells per mL for 143B, HOS, U2, SGC-7901, Hela, BV-2; and 1.8×10⁶ cells per mL for WI-38) were spread on a 6-well plate (1 mL per well), added with a DMEM medium containing 10% fetal calf serum, and cultured overnight to form a monolayer of cells. A horizontal scratch was scratched on the monolayer of cells with a pipette tip of 200 μL, washed with PBS 3 times, added into a DMEM medium containing 10% fetal bovine serum, and then added with corresponding dosages of a quercetin 3,3′-dimethyl ether solution (the dosage of osteosarcoma cells was 143B: 4 mg/L, HOS: 4 mg/L, U2: 10 mg/L; the dosage of breast cancer cells was MDA-MB-231: 2 mg/L, T47D: 3 mg/L; the dosage of gastric cancer cells was SGC7901: 6 mg/L, BGC823: 5 mg/L, AGS: 3 mg/L; the dosage of cervical cancer Hela cells was 2 mg/L; the dosage of lung cancer cell A549 was 3 mg/L; the dosage of intestinal cancer cells Caco2 was 4 mg/L; the dosage of mouse glioma BV2 was 4 mg/L; the dosage of human prostate cancer cells PC3 was 6 mg/L; and the dosage of human thyroid cancer cells BCPAP was 2 mg/L), while DMSO was used as a solvent control, and then incubated for 24 h. A scratch width and a measurement width were determined under a microscope, and a scratch closure ratio was calculated as follows: scratch closure ratio=(scratch width−measurement width)/scratch width×100%. Scratch inhibition ratio=(scratch closure ratio of control group−scratch closure ratio of treatment group)/scratch closure ratio of control group×100%.

The results are shown in Table 2, the quercetin 3,3′-dimethyl ether has a significant inhibitory ability to the scratch closure of 14 kinds of cells. The inhibition rate of scratch closure of osteosarcoma cells is 143B: 54.34%, HOS: 57.60%, U2: 70.46%; the inhibition ratio of breast cancer cells is MDA-MB-231: 81.98%, T47D: 64.12%; the inhibition ratio of gastric cancer cells is SGC7901: 72.02%, BGC823: 73.67%, AGS: 60.91%; the inhibition ratio of cervical cancer Hela cells is 53.97%; the inhibition rate of lung cancer cell A549 was 69.61%; the inhibition rate of Caco2 in intestinal cancer cells is 66.64%; the inhibition ratio of mouse glioma BV2 is 75.22%; the inhibition ratio of human prostate cancer cell PC3 is 70.92%; the inhibition ratio of BCPAP in human thyroid cancer is 64.66%. This shows that the quercetin 3,3′-dimethyl ether treatment has the effect of inhibiting the metastasis of various tumor cells.

TABLE 2
Effect of quercetin 3,3′-dimethyl
ether treatment on scratch closure ratio

| Cell line | Control scratch closed % | Treatment scratch closed % |
|---|---|---|
| 143B | 91.6⁴ + 1.75 | 41.8⁴ + 4.07 * |
| HOS | 92.87 + 4.85 | 39.38 + 4.6⁴ * |
| U2 | 93.68 + 1.28 | 27.67 + 6.81 * |
| MDA-MB-231 | 92.79 + 0.58 | 16.7₂ + 3.87 * |
| T47D | 88.71 + 4.92 | 31.83 + 6.01 * |
| AGS | 81.01 + 4.73 | 31.67 + 3.39 * |
| BGC-823 | 83.7₂ + 3.74 | 22.0⁴ + 9.2₂ * |
| SGC-7901 | 89.53 + 11.04 | 25.05 + 10.1 * |
| Hela | 81.29 + 3.98 | 37.4₂ + 1.6₂ * |
| A549 | 89.80 + 5.11 | 27.29 + 7.7⁴ * |
| Caco2 | 85.0⁴ + 9.49 | 28.37 + 10.21 * |
| BV-2 | 85.4⁴ + 10.33 | 21.17 + 8.55 * |
| PC3 | 85.13 + 9.46 | 24.76 + 5.55 * |
| BCPAP | 85.76 + 7.79 | 30.31 + 5.21 * |

### Example 6

Apoptosis-Promoting Effect of the Quercetin 3,3′-Dimethyl Ether on Tumor Cells

Method:

The tumor cells growing in the logarithmic phase were digested with trypsin, collected by centrifugation to prepare a suspension, the cells were counted and adjusted to a certain concentration (6×10⁵ cells per mL for AGS, T47D, Caco2, BCPAP, GES, HUVEC; 5×10⁵ cells per mL for HepG2, MDA-MB-231, BGC-823, A549, PC3, L02; 3×10⁵ cells per mL for 143B, HOS, U2, SGC-7901, Hela, BV-2; 1.8×10⁶ cells per mL for WI-38), and the cells at a certain concentration were transferred to a 6-well plate at a concentration of 1 mL per well. The cells were cultured in a cell culture incubator for 12 h to allow the cells to adhere to the wall. The corresponding dosages of a quercetin 3,3′-dimethyl ether solution were added (the dosage of osteosarcoma cells was 143B: 4 mg/L, HOS: 4 mg/L, U2: 10 mg/L; the dosage of breast cancer cells was MDA-MB-231: 2 mg/L, T47D: 3 mg/L; the dosage of gastric cancer cells was SGC7901: 6 mg/L, BGC823: 5 mg/L, AGS: 3 mg/L; the dosage of cervical cancer Hela cells was 2 mg/L; the dosage of lung cancer cell A549 was 3 mg/L; the dosage of intestinal cancer cells Caco2 was 4 mg/L; the dosage of mouse glioma BV2 was 4 mg/L; the dosage of human prostate cancer cells PC3 was 6 mg/L; and the dosage of human thyroid cancer cells BCPAP was 2 mg/L). While DMSO was used as a solvent control, and then incubated for 24 h. After acting for 24 h, the cells were collected by centrifugation at 300 g, 4° C. for 5 min, a supernatant was discarded, and the cells were washed twice with pre-cooled PBS, each time with centrifugation at 300 g, 4° C. for 5 min. (1˜5)×10⁵ cells were collected and resuspended in 100 l 1× BindingBuffer. 5 μl of AnnexinV-FITC and 5 μl of PI were added, and mixed well gently. The cells were reacted at room temperature for 10 min in the dark. 400 μl of 1× Binding Buffer was added, mixed well, and the samples were detected by flow cytometry within 1 h. The analysis was conducted according to a standard detection procedure of flow cytometry, at an excitation wavelength of 488 nm, and 20,000 cells were counted, and obtained results were analyzed with cell cycle fitting software ModFit. During analysis, the results were displayed by FL2-w and FL2-A, and the paired cells were removed.

The results are shown in Table 3. The quercetin 3,3′-dimethyl ether has a significant apoptosis-inducing effect on 15 kinds of cells.

TABLE 3
Apoptosis ratio

| | Control apoptosis ratio | Treatment apoptosis ratio |
|---|---|---|
| Cell line | % | % |
| 143B | 5.47 + 2.59 | 24.7₂ + 2.8₂ * |
| HOS | 2.29 + 1.66 | 23.09 + 4.36 * |
| U2 | 5.78 + 1.47 | 27.33 + 4.9⁴ * |
| HepG2 | 3.85 + 0.42 | 32.36 + 5.26 * |
| MDA-MB-231 | 8.05 + 0.67 | 29.85 + 4.49 * |
| T47D | 3.61 + 1.45 | 16.98 + 1.37 * |
| AGS | 5.8₂ + 0.94 | 29.47 + 2.25 * |
| BGC-823 | 7.23 + 1.85 | 26.68 + 3.45 * |
| SGC-7901 | 6.87 + 1.27 | 22.86 + 3.2⁴ * |
| Hela | 6.81 + 1.43 | 20.05 + 4.31 * |
| A549 | 3.73 + 0.93 | 20.28 + 3.23 * |
| Caco2 | 5.3⁴ + 3.65 | 22.81 + 3.7₂ * |
| BV-2 | 2.51 + 0.53 | 18.65 + 0.39 * |
| PC3 | 9.33 + 0.22 | 21.13 + 5.11 * |
| BCPAP | 5.23 + 3.32 | 26.09 + 3.8⁴ * |

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

1. An anti-tumor drug, comprising an oxymethyl-modified derivative of quercetin.

2. The drug according to claim 1, wherein the drug is capable of inhibiting proliferation of a transplanted tumor in vivo.

3. The drug according to claim 1, wherein the drug is capable of inhibiting tumor cell metastasis.

4. The drug according to claim 1, wherein the drug is capable of inducing tumor cell apoptosis.

5. The drug according to claim 1, wherein the oxymethyl-modified derivative of quercetin comprises quercetin 3-methyl ether and/or quercetin 3,3′-dimethyl ether.

6. The drug according to claim 1, wherein the tumor cell is a cell selected from the group consisting of osteosarcoma, liver cancer, breast cancer, gastric cancer, cervical cancer, lung cancer, intestinal cancer, glioma, prostate cancer, and thyroid cancer.

7. A method for preventing or treating tumor based on an oxymethyl-modified derivative of quercetin, comprising the following step: injecting the oxymethyl-modified derivative of quercetin.

8. The method according to claim 7, wherein the oxymethyl-modified derivative of quercetin is added at 12.5 mg/L to 400 mg/L.

9. The method according to claim 7, wherein the oxymethyl-modified derivative of quercetin comprises quercetin 3-methyl ether and/or quercetin 3,3′-dimethyl ether.

10. The method according to claim 7, wherein the tumor is a cell selected from the group consisting of osteosarcoma, liver cancer, breast cancer, gastric cancer, cervical cancer, lung cancer, intestinal cancer, glioma, prostate cancer, and thyroid cancer.

11. The method according to claim 7, comprising inhibiting tumor cell proliferation, inhibiting proliferation of a transplanted tumor in vivo, inhibiting tumor cell metastasis, or inducing tumor cell apoptosis.

12. The drug according to claim 1, wherein the drug is capable of inhibiting tumor cell proliferation.

13. The drug according to claim 5, wherein the drug is capable of inhibiting proliferation of a transplanted tumor in vivo.

14. The drug according to claim 5, wherein the drug is capable of inhibiting tumor cell metastasis.

15. The drug according to claim 5, wherein the drug is capable of inducing tumor cell apoptosis.

16. The drug according to claim 5, wherein the drug is capable of inhibiting tumor cell proliferation.

17. The drug according to claim 6, wherein the drug is capable of inhibiting proliferation of a transplanted tumor in vivo.

18. The drug according to claim 6, wherein the drug is capable of inhibiting tumor cell metastasis.

19. The drug according to claim 6, wherein the drug is capable of inducing tumor cell apoptosis.

20. The drug according to claim 6, wherein the drug is capable of inhibiting tumor cell proliferation.
